(19) United States
(12) Patent Application Publication
(10) Pub. No.: US 2018/0108443 A1
(43) Pub. Date: Apr. 19, 2018

(54) APPARATUS AND METHOD FOR ANALYZING NATURAL LANGUAGE MEDICAL TEXT AND GENERATING A MEDICAL KNOWLEDGE GRAPH REPRESENTING THE NATURAL LANGUAGE MEDICAL TEXT

(71) Applicant: BOE TECHNOLOGY GROUP CO., LTD., Beijing (CN)

(72) Inventor: Hui Li, Beijing (CN)

(73) Assignee: BOE TECHNOLOGY GROUP CO., LTD., Beijing (CN)

(21) Appl. No.: 15/550,557

(22) PCT Filed: Mar. 13, 2017
(86) PCT No.: PCT/CN2017/076439

§ 371 Date: Aug. 11, 2017

(30) Foreign Application Priority Data
Apr. 29, 2016 (CN) 201610281973.6

(51) Int. Cl. G16H 70/20; G06F 17/27; G16H 50/70; G16H 50/20; G06N 5/02

(52) U.S. Cl. CPC G16H 70/20; G06F 17/2785; G06N 5/025; G16H 50/20; G16H 50/70

(57) ABSTRACT

The present application discloses an apparatus for analyzing natural language medical text and generating a medical knowledge graph representing the natural language medical text. The apparatus includes a memory; and one or more processors; the memory and the one or more processors are communicatively connected with each other; the memory gores computer-executable instructions for controlling the one or more processors to acquire a plurality of medical data from a medical data source; extract from the plurality of medical data to obtain a first set of plurality of medical information comprising a first entity of a first entity type and a second entity of a second entity type, a first attribute value of the first entity, a second attribute value of the second entity, and one or more relationships; and generate the medical knowledge graph based on at least a portion of the first set of plurality of medical information.

### CROSS-REFERENCE TO RELATED APPLICATION

This application claims priority to Chinese Patent Application No. 201610281973.6, filed Apr. 29, 2016, the contents of which are incorporated by reference in the entirety.

### TECHNICAL FIELD

The present invention relates to a computer-implemented method for analyzing natural language medical text and generating a medical knowledge graph representing the natural language medical text, a computer-implemented method for querying a medical knowledge graph, and an apparatus for analyzing natural language medical text, and generating a medical knowledge graph representing the natural language medical text.

### BACKGROUND

Knowledge graph is a knowledge base having a graphic structure, the concept of knowledge graph belongs to the category of knowledge engineering. Knowledge graph links knowledge modules of various types and structures from various sources and various disciplines in a graphic format, providing a knowledge system having expandable depth, and breadth based on various meta-data in multiple disciplines. In essence, the knowledge graph integrates knowledge data into a coherent system by establishing relationships among various knowledge modules, and presents the knowledge data in a visual form, e.g., a graphic format. By using various techniques such as data acquisition, data mining, information processing, knowledge measurement, and graphic rendering, the knowledge graph can be used to effectively reveal the dynamic development of a knowledge domain.

### SUMMARY

In one aspect, the present invention provides an apparatus for analyzing natural language medical text and generating a medical knowledge graph representing the natural language medical text, comprising a memory; and one or more processors: wherein the memory and the one or more processors are communicatively connected with each other; the memory stores computer-executable instructions for controlling the one or more processors to acquire a plurality of medical data from a medical data source; extract from the plurality of medical data to obtain a first set of plurality of medical information comprising a first entity of a first entity type and a second entity of a second entity type, a first attribute value of the first entity, a second attribute value of the second entity, and one or more relationships selected from the group consisting of a first relationship between the first entity and the second entity and a second relationship between the first entity and the first attribute value, a third relationship between the first entity and the second attribute value, and a fourth relationship between the second entity and the second attribute value; and generate the medical knowledge graph based on at least a portion of the first set of plurality of medical information.

Optionally, the memory stores computer-executable instructions for controlling the one or more processors to validate the first set of plurality of medical information based on one or more validation criteria to obtain a first set of plurality of validated medical information; and generate the medical knowledge graph based on the plurality of validated medical information.

Optionally, the memory stores computer-executable instructions for controlling the one or more processors to validate the first set of plurality of medical information based on the one or more validation criteria to obtain the first set of plurality of validated medical information and a first set of plurality of invalidated medical information; re-extract a sub-set of the plurality of medical data corresponding to the first set of plurality of invalidated medical information to obtain a second set of plurality of medical information; and re-validate the second, set of plurality of medical information based on the one or more validation criteria to obtain a second set of plurality of validated medical information.

Optionally, the memory stores computer-executable instructions for controlling the one or more processors to reiterate re-extracting and re-validating until all extracted medical information are validated; and generate the medical knowledge graph based on a combination of medical information validated in each round of validation process.

Optionally, the memory stores computer-executable instructions for controlling the one or more processors to store at least a portion of the first set of plurality of medical information in a two-dimensional table subsequent to extracting from the plurality of medical data the first set of plurality of medical information.

Optionally, the memory stores computer-executable instructions for controlling the one or more processors to convert the two-dimensional table into a plurality of graph data; and generate the medical knowledge graph based on the plurality of graph data.

Optionally, the medical data source comprises a medical guideline.

Optionally, the medical knowledge graph comprises a plurality of nodes and one or more edges connecting the plurality of nodes; the plurality of nodes represent a plurality of entities or one or more attribute values of the plurality of entities; and the at least one edge represents one or more relationships between two of the plurality of nodes.

Optionally, the one or more edges are directional edges.

In another aspect, the present invention provides a computer-implemented method for analyzing natural language medical text and generating a medical knowledge graph representing the natural language medical text, comprising acquiring a plurality of medical data from a medical data source; extracting from the plurality of medical data to obtain a first set of plurality of medical information comprising a first entity of a first entity type and a second entity of a second entity type, a first attribute value of the first entity, a second attribute value of the second entity, and one or more relationships selected from the group consisting of a first relationship between the first entity and the second entity and a second relationship between the first entity and the first attribute value, a third relationship between the first entity and the second attribute value, and a fourth relationship between the second entity and the second attribute value; and generating the medical knowledge graph based on at least a portion of the first set of plurality of medical information.

Optionally, generating the medical knowledge graph based on the at least a portion of the first set of plurality of medical information comprises validating the first set of plurality of medical information based on one or, more validation criteria to obtain a first set of plurality of validated medical information; and generating the medical knowledge graph based on the plurality of validated medical information.

Optionally, validating the first set of plurality of medical information comprising validating the first set of plurality of medical information based on one or more validation criteria to obtain the first set of plurality of validated medical information and a first set of plurality of invalidated medical information; the method further comprising re-extracting a sub-set of the plurality of medical data corresponding to the first set of plurality of invalidated medical information to obtain a second set of plurality of medical information; and re-validating the second set of plurality of medical information based on the one or more validation criteria to obtain a second set of plurality of validated medical information.

Optionally, the computer-implemented method further comprises reiterating re-extracting and re-validating until all extracted medical information are validated; and generating the medical knowledge graph based on a combination of medical information validated in each round of validation process.

Optionally, subsequent to extracting from the plurality of medical data the first set of plurality of medical information, the computer-implemented method further comprises storing at least a portion of the first set of plurality of medical information in a two-dimensional table.

Optionally, the computer-implemented method further comprises converting the two-dimensional table into a plurality of graph data; wherein generating the medical knowledge graph comprises generating the medical knowledge graph based on the plurality of graph data.

Optionally, the medical data source comprises a medical guideline.

Optionally, the medical knowledge graph comprises a plurality of nodes and one or more, edges connecting the plurality of nodes; the plurality of nodes represent a plurality of entities or one or more attribute values of the plurality of entities; and the at least one edge represents one or more relationships between two of the plurality of nodes.

Optionally, the one or more edges are directional edges.

In another aspect, the present invention provides a computer-implemented method, comprising receiving a search query comprising a keyword; analyzing the keyword to obtain information regarding one or both of an entity and an attribute value of the entity indicated by the keyword; determining from a medical knowledge graph a knowledge graph data related to the information regarding the one or both of the entity and the attribute value of the entity indicated by the keyword; and causing to be presented representations of the knowledge graph data related to the information regarding the one or both of the entity and the attribute value of the entity indicated by the keyword.

Optionally, the knowledge graph data related to the information regarding the entity or the attribute value of the entity indicated by the keyword comprises one or a combination of data selected from the group consisting of one or more knowledge graph data of an entity associated with the entity indicated by the keyword; one or more knowledge graph data of an attribute value associated with the entity indicated by the keyword; one or more knowledge graph data of an entity associated with the attribute value indicated by the keyword; one or more knowledge graph data of an attribute value associated with the attribute value indicated by the keyword; one or more knowledge graph data of an internet data associated with the entity indicated by the keyword; and one or more knowledge graph data of an internet data associated with the attribute value indicated by the keyword.

### BRIEF DESCRIPTION OF THE FIGURES

The following drawings are merely examples for illustrative purposes according to various disclosed embodiments and are not intended to limit the scope of the present invention.

FIG. 1 is a flow chart illustrating a method of generating a medical knowledge graph in some embodiments according to the present disclosure.

FIG. 2 is a schematic representation of a medical knowledge graph in some embodiments according to the present disclosure.

FIG. 3 is a flow chart illustrating a method of querying a medical knowledge graph in some embodiments according to the present disclosure.

FIG. 4 is a schematic diagram illustrating an apparatus for querying a medical knowledge graph in some embodiments according to the present disclosure.

FIG. 5 is schematic diagram illustrating the structure of an apparatus for generating a medical knowledge graph in some embodiments according to the present disclosure.

### DETAILED DESCRIPTION

The disclosure will now be described more specifically with reference to the following embodiments. It is to be noted that the following descriptions of some embodiments are presented herein for purpose of illustration and description only. It is not intended to be exhaustive or to be limited to the precise form disclosed.

Knowledge graph has been used to in various application settings such as search engines to enhance the search engine's search results with semantic-search information gathered from various sources in conventional knowledge graphs, a relational data structure is used in building the knowledge graph. Knowledge graph, however, has not been used in medical knowledge applications, in the medical field, various medical data contains complicated relationships between diseases, symptoms, and therapies. The relational data structure used in conventional knowledge graph is not suitable for data mining and data expansion in a medical knowledge graph, and is incapable of providing an intuitive reference tool for a user.

Accordingly, the present invention provides, inter alia, a computer-implemented method for analyzing natural language medical text and generating a medical knowledge graph representing the natural language medical text, a computer-implemented method for querying a medical knowledge graph, and an apparatus for analyzing natural language medical text and generating a medical knowledge graph representing the natural language medical text that substantially obviate one or mare of the problems due to limitations and disadvantages of the related art. In one aspect, the present disclosure provides a computer-implemented method for analyzing natural language medical text and generating a medical knowledge graph representing the natural language medical text. In some embodiments, the method includes acquiring a plurality of medical data from a medical data source; extracting from the plurality of medical data to obtain a first set of plurality of medical information including a first entity of a first entity type and a second entity of a second entity type, a first attribute value of the first entity, a second attribute value of the second entity, and one or more relationships selected from the group consisting of a first relationship between the first entity and the second entity and a second relationship between the first entity and the first attribute value, a third relationship between the first entity and the second attribute value, and a fourth relationship between the second entity and the second attribute value; and generating the medical knowledge graph based on at least a portion of the first set of plurality of medical information. Optionally, the method includes acquiring a plurality of medical data from a medical data source; extracting from the plurality of medical data to obtain a first set of plurality of medical information including a plurality of entities, a plurality of attribute values, and a plurality of relationships between the entities; and generating the medical knowledge graph based on at least a portion of the first set of plurality of medical information.

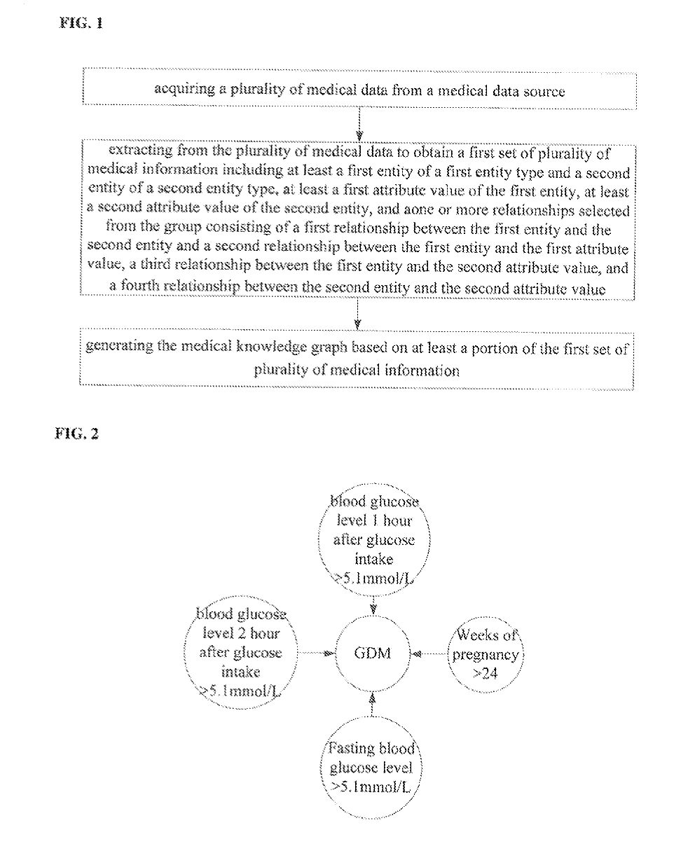

FIG. 1 is a flow chart illustrating a method of generating a medical knowledge graph in some embodiments according to the present disclosure. The method analyzes natural language medical text and generates a medical knowledge graph representing the natural language medical text. Referring to FIG. 1, the method in some embodiments includes acquiring a plurality of medical data from a medical data source; extracting from the plurality of medical data to obtain a first set of plurality of medical information including at least a first entity of a first entity type and a second entity of a second entity type, at least a first attribute value of the first entity, at least a second attribute value of the second entity, and one or more relationships selected from the group consisting of a first relationship between the first entity and the second entity and a second relationship between the first entity and the first attribute value, a third relationship between the first entity and the second attribute value, and a fourth relationship between the second entity and the second attribute value; and generating the medical knowledge graph based on at least a portion of the first set of plurality of medical information.

In the present method, a medical knowledge graph is constructed based on entities, attribute values of the entities, and relationships between various entities. By generating a medical knowledge graph, a great amount of natural language medical texts can be represented in a much-simplified form. Various characteristics and attributes of various entities, and their correlations can be directly visualized in the medical knowledge graph. A database having the present medical knowledge graph can serve as an intuitive and convenient reference for medical practitioner, thereby reducing the occurrence of medical malpractice.

In some embodiments, the medical knowledge graph is generated based on, a plurality of validated medical information. Accordingly, the step of generating the medical knowledge based on the at least a portion of the first set of plurality of medical information includes validating the first set of plurality of medical information based on one or more validation criteria to obtain a first set of plurality of validated medical information; and generating the medical knowledge graph based on the plurality of validated medical information, e.g., when the first set of plurality of medical information is validated. In one example, the first set of plurality of validated medical information includes one or a combination of validated medical information selected from the group consisting of at least a first validated entity of a first entity type and a second validated entity of a second entity type, at least a first validated attribute value of the first entity, at least a second validated attribute value of the second entity, and one or more validated relationships selected from the group consisting of a first validated relationship between the first entity and the second entity and a second validated relationship between the first entity and the first attribute value, a third validated relationship between the first entity and the second attribute value, and a fourth validated relationship between the second entity and the second attribute value. Optionally, the first set of plurality of validated medical information includes one or a combination of validated medical information selected from the group consisting of a plurality of validated entities, a plurality of validated attribute values, and a plurality of validated relationships between the entities.

In the medical field, it is important to ensure reliability and integrity of any medical data to be used as a reference tool. Various validation methods may be implemented. In some embodiments, the extracted medical information is validated manually, e.g., by a medical doctor or an expert in a particular medical field. In some embodiments, the extracted medical information may he validated by comparing the extracted medical information with medical information extracted from other medical data sources. Optionally, the extracted medical information is validated if the extracted medical information is substantially consistent with medical information extracted from a plurality of medical data sources.

In some embodiments, the validation process produces a validated set of medical information and an invalidated set of medical information. Optionally, the validation process produces a first set of plurality of validated medical information and a first set of plurality of invalidated medical information. Optionally, the first set of plurality of validated medical information includes at least a first validated entity of a first entity type and a second validated entity of a second entity type, at least a first validated attribute value of the first entity, at least a second validated attribute value of the second entity, and one or more validated relationships selected from the group consisting of a first validated relationship between the first entity and the second entity and a second validated relationship between the first entity and the first attribute value, a third validated relationship between the first entity and the second attribute value, and a fourth validated relationship between the second entity and the second attribute value. Optionally, the first set of plurality of validated medical information includes a plurality of validated entities, a plurality of validated attribute values, and a plurality of validated relationships between the entities. Optionally, the first set of plurality of invalidated medical information includes one or a combination of medical information selected from the group consisting of one or more invalidated entities, one or more invalidated attribute values, and one or more invalidated relationships selected from the group consisting of a first invalidated relationship between the first entity and the second entity and a second invalidated relationship between the first entity and the first attribute value, a third invalidated relationship between the first entity and the second attribute value, and a fourth invalidated relationship between the second entity and the second attribute value. Accordingly, in some embodiments, the step of validating the first set of plurality of medical information includes validating the first set of plurality of medical information based on one or more validation criteria to obtain the first set of plurality of validated medical information and the first set of plurality of invalidated medical information. The method further includes re-extracting a sub-set of the plurality of medical data corresponding to the first set of plurality of invalidated medical information to obtain a second set of plurality of medical information; and re-validating the second set of plurality of medical information based on the one or more validation criteria to obtain a second set of plurality of validated medical information. If all information in the second set of plurality of medical information are validated, the second set of plurality of validated medical information may be combined with the first set of plurality of validated medical information to generate the medical knowledge graph. If some information in the second set of plurality of medical information are still being invalidated, the re-extracting and re-validating processes are repeated until all medical information are validated. In one example, the method further includes reiterating the re-extracting and re-validating processes until all extracted medical information are validated, and generating the medical knowledge graph based on a combination of medical information validated in each round of validation process. Optionally, the combination of medical information validated in each round of validation process includes at least a first validated entity of a first entity type and a second validated entity of a second entity type, at least a first validated attribute value of the first entity, at least a second validated attribute value of the second entity, and one or more validated relationships selected from the group consisting of a first validated relationship between the first entity and the second entity and a second validated relationship between the first entity and the first attribute value, a third validated relationship between the first entity and the second attribute value, and a fourth validated relationship between the second entity and the second attribute value. Optionally, the combination of medical information validated in each round of validation process includes a plurality of validated entities, a plurality of validated attribute values, and a plurality of validated relationships between the entities.

In some embodiments, the method Rather includes storing a plurality of medical information used for generating the medical knowledge graph in a two-dimensional table. In one example, the plurality of medical information used for generating the medical knowledge graph is the first set of plurality of medical information. In another example, the plurality of medical information used for generating the medical knowledge graph is the first set of plurality of validated medical information. In another example, the plurality of medical information used for generating the medical knowledge graph is the combination of medical information validated in each round of validation process. In another example, the plurality of medical information used for generating the medical knowledge graph include a plurality of validated entities, a plurality of validated attribute values, and a plurality of relationships between various entities and various attribute values. Optionally, the two-dimensional table is in a form of a spreadsheet such as an excel spreadsheet. The two-dimensional table may be generated using an automatic extraction method such as a supervised algorithm (e.g., svm, maxent), an unsupervised algorithm (e.g., bootstrapping), and distant supervision.

Optionally, subsequent to extracting from the plurality of medical data the first set of plurality of medical information, the method further includes storing at least a portion of the first set of plurality of medical information in a two-dimensional table.

The plurality of medical information used for generating the medical knowledge graph may be stored in a form other than the two-dimensional table. For example, the plurality of medical information used for generating the medical knowledge graph may be stored in a tree form. By first representing the medical information in a simplified form to highlight the essential contents of the medical data, the medical information may be easily converted into a graph form, facilitating the generation of the medical knowledge graph, which serves as a useful reference tool in an intuitive format.

In some embodiments, the plurality of medical information stored in the two-dimensional table is further converted into a plurality of graph data. The method in some embodiments further includes converting the two-dimensional table into a plurality of graph data, and the step of generating the medical knowledge graph includes generating the medical knowledge graph based on the plurality of graph data. In the context of the present disclosure, the term “graph data” refers to data representing a graph including a plurality of nodes and one or more edges connecting respective nodes. Nodes in the graph may be of the same or different types.

In one example, the entity may be a disease, a therapy for treating a disease, a diagnosis method for diagnosing a disease, a prognosis method for a disease, a drug for treating or preventing a disease, etc. In another example, the attribute value may be a symptom of a disease, a clinical manifestation of a disease, diagnosis information, etc. In another example, the relationship between an entity and an attribute value may be an association between two diseases, a correlation between two diseases, and so on. By converting the medical information in a table form into a graph data form, the medical information may be represented in a non-relational data storage form, which is more conducive to data expansion and data mining. The data in a graph data form offers an intuitive and convenient viewing tool for a user. The medical information in the table format may be convened into the graph data format by various appropriate methods such as a computer program.

In one example, the medical data source contains the following natural language medical text:

“Gestational diabetes (GDM) refers to abnormal glucose metabolism during pregnancy. When a blood glucose level of a patient is found to he higher than to equal to a diabetic level for the first time during pregnancy, it should be properly diagnosed as pre-gestational diabetes (PGDM) instead of gestational diabetes. The GDM diagnostic methods and standards are as follow:

It is recommended that a medical institution should conduct an oral glucose tolerance test (OGTT) on a first visit of a pregnant woman who has not been diagnosed as PGDM or GDM, during 24 to 28 weeks of pregnancy or after 28 weeks of pregnancy.

75 g OGTT method: the patient should fast for at least 8 hours continuously before the OGTT test. The patient should have a normal diet for continuously three days before the OGTT test with no less than 150 g daily carbohydrate intake. During the OGTT test, the patient should sit still, and should not be allowed to smoke. In the OGTT test, 300 ml of a solution containing 75 g glucose is administered to the patient orally. Venous blood of the patient before the glucose administration, 1 hour after the glucose administration, and 2 hour glucose administration (starting from a time point when the glucose is administered) are taken. Each blood sample is placed into a test tube containing sodium fluoride. The blood glucose level is determined using a glucose oxidase method.

75 g OGTT diagnosis standard: Blood glucose levels before the glucose administration, 1 hour after the glucose administration, and 2 hour glucose administration should be respectively lower than 5.1 nmol (92 mg/dl), 10.0 mmol/L (180 mg/dl), and 8.5 mmol/L (153 mg/dl). GDM is properly diagnosed if any of three blood glucose level is above a threshold level.”

The plurality of medical information extracted from the above medical data source may be represented in the following two-dimensional table:

| Condition | | Conclusion |
|---|---|---|
| Weeks of pregnancy | >24 | GDM |
| Fasting blood glucose level | >5.1 mmol/L | |
| blood glucose level 1 hour | >10.0 mmol/L | |
| after glucose intake | | |
| Wood glucose level 2 hour | >8.5 mmol/L | |
| after glucose intake | | |

In the example, the disease GDM is considered a first entity of a first type, and the diagnosis standard is considered as a second entity of a second entity type. For example, each of the conditions listed in the above table may be considered as an entity. When one or more conditions for GDM in the table are met, the diagnosis conclusion of GDM is made.

The entities, the attribute values, and the relationships between entities and attribute values in the two-dimensional table can he converted into graph data by a computer program for generating a medical knowledge graph. As compared to other data format having, a relational data structure, the graph data has a non-relational data structure, which can be used to better represent diverse types of relationships among various data, and is conducive to data expansion and data mining. The knowledge graph generated using the graph data according to the present disclosure can be used in a multi-directional knowledge mining process, and provides a more intuitive reference tool to medical practitioners, thereby reducing the occurrence of medical malpractice.

Various reliable medical data sources may be used in the present method. Optionally, the medical data source includes a medical guideline. Examples of appropriate medical data sources includes a medical encyclopedia, a clinical practice guideline, and a medical textbook.

In some embodiments, the medical knowledge graph includes a plurality of nodes and at least one edge connecting the plurality of nodes. The plurality of nodes represent a plurality of entities or one or more attribute values of the plurality of entities. The at least one edge represents at least one relationship between two of the plurality of nodes. Optionally, the at least one edge is a directional edge.

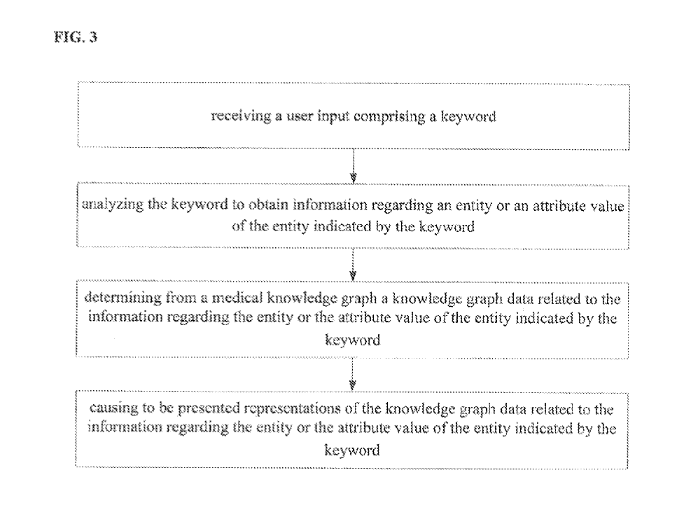

FIG. 2 is a schematic representation of a medical knowledge graph in some embodiments according to the present disclosure. Referring to FIG. 2, the disease (diagnosis conclusion) GDM, and the conditions for making a GDM diagnosis, are used as nodes of the medical knowledge graph. The relationships between GDM and the conditions for making a GDM diagnosis are used as directional edges of the medical knowledge graph.

In some embodiments, a disease, its symptom, and its therapy are used as nodes of the medical knowledge graph, and the relationship between the disease and its symptom and the relationship between the disease and its therapy are used as directional edges of the medical knowledge graph. Additional nodes representing other related concepts or entities may be included in the medical knowledge graph.

In another aspect, the present disclosure provides a computer-implemented method for querying a medical knowledge graph such as a medical knowledge graph described in the present disclosure or generated by a method described in the present disclosure. FIG. 3 is a flow chart illustrating a method of querying a medical knowledge graph in some embodiments according to the present disclosure. Referring to FIG. 3, in some embodiments, the method includes receiving a user input including a keyword; analyzing the keyword to obtain information regarding an entity or an attribute value of the entity indicated by the keyword; determining from a medical knowledge graph a knowledge graph data related to the information regarding the entity or the attribute value of the entity indicated by the keyword; and causing to be presented representations of the knowledge graph data related to the information regarding the entity or the attribute value of the entity indicated by the keyword, e.g., to a user.

In some embodiments, the knowledge graph data related to the information regarding the entity or the attribute value of the entity indicated by the keyword includes one or a combination of data such as one or more knowledge graph data of an entity associated with the entity indicated by the keyword; one or more knowledge graph data of an entity associated with the attribute value indicated by the keyword; one or more knowledge graph data of an attribute value associated with the attribute value indicated by the keyword; one or more knowledge graph data of an interact data associated with the entity indicated by the keyword; and one or more knowledge graph data of an interact data associated with the attribute value indicated by the keyword.

In one example, the keyword inputted by a user is a name of a disease, or a symptom. The medical knowledge graph is queried, the knowledge graph data related to the disease or its symptom is determined in the medical knowledge graph, and the determined knowledge graph data is displayed to the user.

In another aspect, the present disclosure provides a computer-implemented method for querying a medical knowledge graph such as a medical knowledge graph described in the present disclosure or generated by a method described in the present disclosure. In some embodiments, the method includes receiving a clinical data; analyzing the clinical data by applying a set of rules on the clinical data and generating a search query; analyzing the search query (e.g., a keyword) to obtain information regarding an entity or an attribute value of the entity indicated by the search query; determining from the medical knowledge graph a knowledge graph data related to the information regarding the entity or the attribute value the entity indicated by the search query; arid causing to be presented representations of the knowledge graph data related to the information regarding the entity or the attribute value of the entity indicated by the search query, e.g., to a user. In some embodiments, the method includes receiving, at a decision support system, a clinical data; transmitting the clinical data from the decision support system to a rule engine; analyzing the clinical data by the rule engine applying a set of rules on the clinical data and generating a search query; transmitting, from the rule engine, the search query to the decision support system; transmitting, from the decision support system, the search query to a medical knowledge graph; analyzing the search query (e.g., a keyword) to obtain information regarding an entity or an attribute value of the entity indicated by the search query; determining from the medical knowledge graph a knowledge graph data related to the information regarding the entity or the attribute value of the entity indicated by the search query; and causing to be presented representations of the knowledge graph data related to the information regarding the entity or the attribute value of the entity indicated by the search query, e.g., to a user. Optionally, the method further includes recommending a therapy to the user. Optionally, the decision support system includes a Drools business process management system.

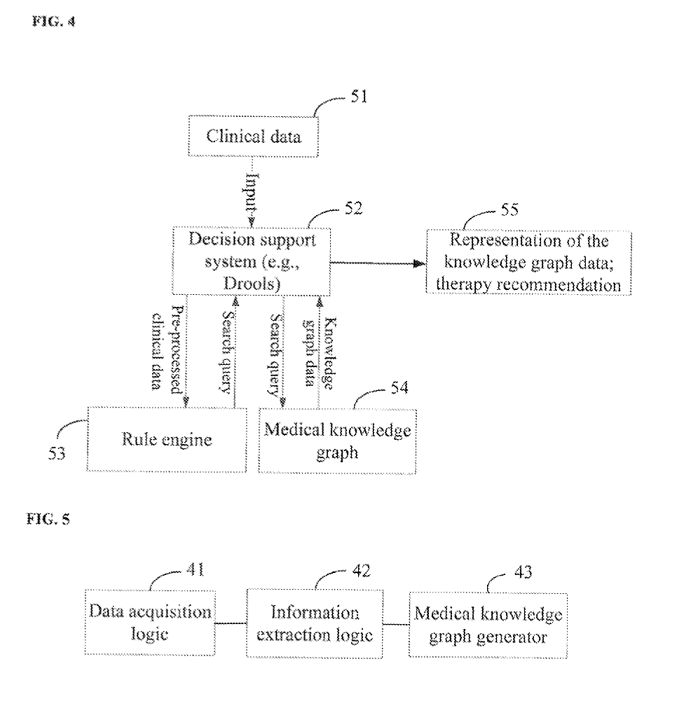

In another aspect, the present disclosure provides an apparatus for querying a medical knowledge graph such as a medical knowledge graph described in the present disclosure or generated by a method described in the present disclosure. In some embodiments, the apparatus includes a decision support system configured to receive a clinical data, process the clinical data; a rule engine configured to receive a processed clinical data from the decision support system, apply a set of rules on the processed clinical data to generate a search query, and transmit the search query to the decision support system; a medical knowledge graph configured to receive the search query from the decision support system, analyze the search query (e.g., a keyword) to obtain information regarding an entity or an attribute value of the entity indicated by the search query, determine from the medical knowledge graph a knowledge graph data related to the information regarding the entity or the attribute value of the entity indicated by the search query; wherein the decision support system is configured to receive the knowledge graph data related to the information regarding the entity or the attribute value of the entity indicated, by the search query, and present representations of the knowledge graph data related to the information regarding the entity or the attribute value of the entity indicated by the search query, e.g., to a user. Optionally, the decision support system is further configured to recommend a therapy to the user. Optionally, the decision support system includes a Drools business process management system. FIG. 4 is a schematic diagram illustrating an apparatus for querying a medical knowledge graph in some embodiments according to the present disclosure.

In another aspect, the present disclosure provides an apparatus for analyzing natural language medical text and generating a medical knowledge graph representing the natural language medical text. In some embodiments, the apparatus includes a memory; and one ok more processors, the memory and the at least one processor being communicatively connected with each other. The memory stores computer-executable instructions for controlling the one or more processors to acquire a plurality of medical data from a medical data source; extract from the plurality of medical data to obtain a first set of plurality of medical information including at least a first entity of a first entity type and a second entity of a second entity type, at least a first attribute value of the first entity, at least a second attribute value of the second entity, and one or more validated relationships selected from the group consisting of a first validated relationship between the first entity and the second entity and a second validated relationship between the first entity and the first attribute value, a third validated relationship between the first entity and the second attribute value, and a fourth validated relationship between the second entity and the second attribute value; and generate the medical knowledge graph based on at least a portion of the first set of plurality of medical information. Optionally, the first set of plurality of medical information includes a plurality of entities, a plurality of attribute values, and a plurality of relationships between entities. Optionally, the memory stores computer-executable instructions for controlling the one or more processors to validate the first set of plurality of medical information based on one or more validation criteria to obtain a first set of plurality of validated medical information; and generating the medical knowledge graph based on the plurality of validated medical information, e.g., when the first set of plurality of medical information is validated. Optionally, the memory stores computer-executable instructions for controlling the one or more processors to validate the first set of plurality of medical information based on one or more validation criteria to obtain the first set of plurality of validated medical information and a first set of plurality of invalidated medical information; re-extract a sub-set of the plurality of medical data corresponding to the first set of plurality of invalidated medical information to obtain a second set of plurality of medical information; and re-validate the second set of plurality of medical information based on the one or more validation criteria to obtain a second set of plurality of validated medical information. Optionally, the memory stores computer-executable instructions for controlling the one or more processors to reiterate re-extracting and re-validating until all extracted medical information are validated; and generate the medical knowledge graph based on a combination of medical information validated in each round of validation process.

In some embodiments, the memory stores computer-executable instructions for controlling the one or more processors to store at least a portion of the first set of plurality of medical information in a two-dimensional table subsequent to extracting from the plurality of medical data the first set of plurality of medical information. Optionally, the memory stores computer-executable instructions for controlling the one or more processors to converting the two-dimensional table into a plurality of graph data; and generating the medical knowledge graph based on the plurality of graph data.

Optionally, the medical data source includes a medical guideline.

In some embodiments, the medical knowledge graph includes a plurality of nodes and at least one edge connecting the plurality of nodes. The plurality of nodes represent a plurality of entities or one or more attribute values of the plurality of entities. The at least one edge represents at least one relationship between two of the plurality of nodes. Optionally, the at least one edge is a directional edge.

FIG. 5 is schematic diagram illustrating the structure of an apparatus for generating a medical knowledge graph in some embodiments according to the present disclosure. Referring to FIG. 5, the apparatus in some embodiments includes a data acquisition logic 41 for acquiring a plurality of medical data from a medical data source; an information extraction logic 42 for extracting from the plurality of medical data to obtain a first set of plurality of medical information including at least a first entity of a first entity type and a second entity of a second entity type, at least a first attribute value of the first entity, at least a second attribute value of the second entity, and one or more validated relationships selected from the group consisting of a first validated relationship between the first entity and the second entity and a second validated relationship between the first entity and the first attribute value, a third validated relationship between the first entity and the second attribute value, and a fourth validated relationship between the second entity and the second attribute value; and a medical knowledge graph generator 43 for generating the medical knowledge graph based on at least a portion of the first set of plurality of medical information.

As used herein, the term “logic” refers to hardware (e.g. a board, circuit, chip, etc.), software and/or firmware configured to carry out operations according to the invention. For instance, features of the invention may be accomplished by specific circuits under control of a computer program or program modules stored on a suitable computer-readable medium, where the program modules are configured to control the execution of memory operations using the circuitry of the interface.

In some embodiments, the medical knowledge graph generator 43 is configured to validate the first set of plurality of medical information based on one or more validation criteria to obtain a first set of plurality of validated medical information; and configured to generate the medical knowledge graph based on the plurality of validated medical information.

In some embodiments, the medical knowledge graph generator 43 is configured to validate the first set, of plurality of medical information based on one or more validation criteria to obtain the first set of plurality of validated medical information and a first set of plurality of invalidated medical information. Optionally, the information extraction logic 42 is configured to re-extract a sub-set of the plurality of medical data corresponding to the first set of plurality of invalidated medical information to obtain a second set of plurality of medical information; and configured to re-validate the second set of plurality of medical information based on the one or more validation criteria to obtain a second set of plurality of validated medical information. Optionally, the information extraction logic 42 is configured to reiterate the re-extracting and re-validating processes until all extracted medical information are validated, and the medical knowledge graph generator 43 is configured to generate the medical knowledge graph based on a combination of medical information validated in each round of validation process.

In some embodiments, the information extraction logic 42 is configured to store at least a portion of the first set of plurality of medical information in a two-dimensional table. Optionally, the information extraction logic 42 is configured to store at least a portion of the first set of plurality of medical information in a two-dimensional table subsequent to extracting from the plurality of medical data the first set of plurality of medical information.

In some embodiments, the medical knowledge graph generator 43 is configured to convert the two-dimensional table into a plurality of graph data; and generate the medical knowledge graph based on the plurality of graph data.

Optionally, the data acquisition logic 41 is configured to acquire a plurality of medical data from a medical guideline.

In some embodiments, the medical knowledge graph generator 43 is configured to generate a medical knowledge graph having a plurality of nodes and at least one edge connecting the plurality of nodes. The plurality of nodes represent a plurality of entities or one or more attribute values of the plurality of entities. The at least one edge represents at least one relationship between two of the plurality of nodes. Optionally, the at least one edge is a directional edge.

The present disclosure provides, inter alia, a method for generating a medical knowledge graph, a method for querying the medical knowledge graph, and an apparatus for generating a medical knowledge graph. The present method and apparatus acquire medical data from a medical data source, extract from the medical data a plurality of medical information including entities, attribute values, and relationships between entities, and generate medical knowledge graph based on the plurality of medical information. The medical information is stored in a non-relational data storage format, which can he used in a multi-directional knowledge mining process, and provides a more intuitive reference tool to medical practitioners, thereby reducing the occurrence of medical malpractice

The foregoing description of the embodiments of the invention has been presented for purposes of illustration and description. It is not intended to be exhaustive or to limit the invention to the precise form or to exemplary embodiments disclosed. Accordingly, the foregoing description should be regarded as illustrative rather than restrictive. Obviously, many modifications and variations will be apparent to practitioners skilled in this art. The embodiments are chosen and described in order to explain the principles of the invention and its best mode practical application, thereby to enable persons skilled in the art to understand the invention for various embodiments and with various modifications as are suited to the particular use or implementation contemplated. It is intended that the scope of the invention be defined by the claims appended hereto and their equivalents in which all terms are meant in their broadest reasonable sense unless otherwise indicated. Therefore, the term “the invention”, “the present invention” or the like does not necessarily limit the claim scope to a specific embodiment, and the reference to exemplary embodiments, of the invention does not imply a limitation on the invention, and no such limitation is to be inferred. The invention is limited only by the spirit and scope of the appended claims. Moreover, these claims may refer to use “first”, “second”, etc. following with noun or element. Such terms should be understood as a nomenclature and should not be construed as giving the limitation on the number of the elements modified by such nomenclature unless specific number has been given. Any advantages and benefits described may not apply to all embodiments of the invention. It should be appreciated that variations may be made in the embodiments described by persons skilled in the art without departing from the scope of the present invention as defined by the following claims. Moreover, no element and component in the present disclosure is intended to he dedicated to the public regardless of whether the element or component is explicitly recited in the following claims.

1. An apparatus for analyzing natural language medical text and generating a medical knowledge graph representing the natural language medical text, comprising:
a memory; and
one or more processors;
wherein the memory and the one or more processors are communicatively connected with each other;
the memory stores computer-executable instructions for controlling the one or more processors to:
acquire a plurality of medical data from a medical data source;
extract from the plurality of medical data to obtain a first set of plurality of medical information comprising a first entity of a first entity type and a second entity of a second entity type, a first attribute value of the first entity, a second attribute value of the second entity, and one or more relationships selected from the group consisting, of a first relationship between the first entity and the second entity and a second relationship between the first entity and the first attribute value, a third relationship between the first entity and the second attribute value, and a fourth relationship between the second entity and the second attribute value; and
generate the medical knowledge graph based on at least a portion of the first set of plurality of medical information.

2. The apparatus of claim I, wherein the memory stores computer-executable instructions for controlling the one or more processors to:
validate the first set of plurality of medical information based on one or more validation criteria to obtain a first set of plurality of validated medical information; and
generating the medical knowledge graph based on the plurality a validated medical information.

3. The apparatus of claim 2, wherein the memory stores computer-executable instructions for controlling the one or more processors to:
validate the first set of plurality of medical information based on the one or more validation criteria to obtain the first set of plurality of validated medical information and a first set of plurality of invalidated medical information;
re-extract a sub-set of the plurality of medical data corresponding to the first set of plurality of invalidated medical information to obtain a second set of plurality of medical information; and
re-validate the second set of plurality of medical information based on the one or more validation criteria to obtain a second set of plurality of validated medical information.

4. The apparatus of claim 3, wherein the memory stores computer-executable instructions for controlling the one or more processors to:
reiterate re-extracting and re-validating until all extracted medical information are validated; and
generate the medical knowledge graph based on a combination of medical information validated in each round of validation process.

5. The apparatus of claim 1, the memory stores computer-executable instructions for controlling the one or more processors to:
store at least a portion of the first set of plurality of medical information in a two-dimensional table subsequent to extracting from the plurality of medical data the first set of plurality of medical information.

6. The apparatus of claim 5, the memory stores computer-executable instructions for controlling the one or more processors to:
converting the two-dimensional table into a plurality of graph data; and
generating the medical knowledge graph based on the plurality of graph data.

7. The apparatus of claim 1, wherein the medical data source comprises a medical guideline.

8. The apparatus of claim 1, wherein the medical knowledge graph comprises a plurality of nodes and one or more edges connecting the plurality anodes;
the plurality of nodes represent a plurality of entities or one or more attribute values of the plurality of entities; and
the at least one edge represents one or more relationships between two of the plurality of nodes.

9. The apparatus of claim 8, wherein the one or more edges are directional edges.

10. A computer-implemented method for analyzing natural language medical text and generating a medical knowledge graph representing the natural language medical text, comprising:
acquiring a plurality of medical data from a medical data source;
extracting from the plurality of medical data to obtain a first set of plurality of medical information comprising a first entity of a first entity type and a second entity of a second entity type, a first attribute value of the first entity, a second attribute value of the second entity, and one or more relationships selected from the group consisting of a first relationship between the first entity and the second entity and a second relationship between the first entity and the first attribute value, a third relationship between the first entity and the second attribute value, and a fourth relationship between the second entity and the second attribute value; and
generating the medical knowledge graph based on at least a portion of the first set of plurality of medical information.

11. The computer-implemented method of claim 10, wherein generating the medical knowledge graph based on the at least a portion of the first set of plurality of medical information comprises:
validating the first set of plurality of medical information based on one or more validation criteria to obtain a first set of plurality of validated medical information; and
generating the medical knowledge graph based on the plurality of validated medical information.

12. The computer-implemented method of claim 11, wherein validating the first set of plurality of medical information comprising validating the first set of plurality of medical information based on one or more validation criteria to obtain the first set of plurality of validated medical information and a first set of plurality of invalidated medical information;
the method further comprising:
re-extracting a sub-set of the plurality of medical data corresponding to the first set of plurality of invalidated medical information to obtain a second set of plurality of medical information;
re-validating the second set of plurality of medical information based on the one or more validation criteria to obtain a second set of plurality of validated medical information.

13. The computer-implemented method of claim 12, further comprising:
reiterating re-extracting and re-validating until all extracted medical information are validated; and
generating the medical knowledge graph based on a combination of medical information validated in each round of validation process.

14. The computer-implemented method of claim 10, subsequent to extracting from the plurality of medical data the first set of plurality of medical information, further comprising:
storing at least a portion of the first set of plurality of medical information in a two-dimensional table.

15. The computer-implemented method of claim 14, further comprising converting the two-dimensional table into a plurality of graph data;
wherein generating the medical knowledge graph comprises generating the medical knowledge graph based on the plurality of graph data.

16. The computer-implemented method of claim 10, wherein the medical data source comprises a medical guideline.

17. The computer-implemented method of claim 10, wherein the medical knowledge graph comprises a plurality of nodes and one or more edges connecting the plurality of nodes;
the plurality of nodes represent a plurality of entities or one or more attribute values of the plurality of entities; and
the at least one edge represents one or more relationships between two of the plurality of nodes.

18. The computer-implemented method of claim 17, wherein the one or more edges are directional edges.

19. A computer-implemented method, comprising:
receiving a search query comprising a keyword;
analyzing the keyword to obtain information regarding one or both of an entity and an attribute value of the entity indicated by the keyword;
determining from a medical knowledge graph a knowledge graph data related to the information regarding the one or both of the entity and the attribute value of the entity indicated by the keyword; and
causing to be presented representations of the knowledge graph data related to the information regarding the one or both of the entity and the attribute value of the entity indicated by the keyword.

20. The computer-implemented method of claim 19, wherein the knowledge graph data related to the information regarding the entity or the attribute value of the entity indicated by the keyword comprises one or a combination of data selected from the group consisting of:
one or more knowledge graph data of an entity associated with the entity indicated by the keyword;
one or more knowledge graph data of an attribute value associated with the entity indicated by the keyword;
one or more knowledge graph data of an entity associated with the attribute value indicated by the keyword;
one or more knowledge graph data of an attribute value associated with the attribute value indicated by the keyword;
one or more knowledge graph data of an internet data associated with e entity indicated by the keyword; and
one or more knowledge graph data of an internet data associated with the attribute, value indicated by the keyword.
